(19) United States
(12) Patent Application Publication
(10) Pub. No.: US 2022/0106406 A1
(43) Pub. Date: Apr. 7, 2022

(54) POLYPEPTIDE DIRECTED AGAINST PROTEIN TYROSINE PHOSPHATASE 4A PROTEINS, AND COMPOSITIONS AND METHODS FOR USE THEREOF

(71) Applicant: University of Kentucky Research Foundation, Lexington, KY (US)

(72) Inventors: Jessica S. Blackburn, Harrodsburg, KY (US); Caroline N. Smith, Richmond, KY (US)

(21) Appl. No.: 17/492,299

(22) Filed: Oct. 1, 2021

Related U.S. Application Data
(60) Provisional application No. 63/086,396, filed on Oct. 1, 2020.

(51) Int. Cl. C07K 16/40; C12N 9/10; G01N 33/573

(52) U.S. Cl. CPC C07K 16/40; C12N 9/104; C07K 2319/00; C07K 2317/569; G01N 2333/9108; G01N 33/573

(57) ABSTRACT

A protein tyrosine phosphatase 4A (PTP4A or PRL) targeting amino acid molecule is provided. The PRL targeting amino acid molecule includes an amino acid sequence according to one or more of NB91 (SEQ ID NO: 1), NB 13 (SEQ ID NO: 2), NB90 (SEQ ID NO: 3), NB4 (SEQ ID NO: 4), NB7 (SEQ ID NO: 5), NB10 (SEQ ID NO: 6), NB16 (SEQ ID NO: 7), NB18 (SEQ ID NO: 8), NB29 (SEQ ID NO: 9), NB19 (SEQ ID NO: 10), NB84 (SEQ ID NO: 11), NB92 (SEQ ID NO: 12), NB23 (SEQ ID NO: 13), NB26 (SEQ ID NO: 14), NB28 (SEQ ID NO: 15), NB68 (SEQ ID NO: 16), or a variant thereof. Also provided herein are methods of making and using the PRL targeting amino acid molecule.

### RELATED APPLICATIONS

This application claims the benefit of U.S. Provisional Application Ser. No. 63/086,396, filed Oct. 1, 2020, the entire disclosure of which is incorporated herein by this reference.

### GOVERNMENT INTEREST

This invention was made with government support under grant number CA227656 awarded by the National Institutes of Health. The government has certain rights in the invention.

### SEQUENCE LISTING

The instant application contains a Sequence Listing which has been submitted in ASCII format via EFS-Web and is hereby incorporated by reference in its entirety. The ASCII copy of the Sequence Listing, which was created on Oct. 1, 2021, is named UK2536—Sequence Listing.txt and is 21.8 kilobytes in size.

### TECHNICAL FIELD

The presently-disclosed subject matter generally relates to inhibition of members of the protein tyrosine phosphatase 4A (PTP4A or PRL) family of proteins. In particular, certain embodiments of the presently-disclosed subject matter relate to amino acid sequences or nanobodies specific for PTP4A3 or PRL-3, and use thereof in research and therapeutic methods, including in the context of cancer.

### BACKGROUND

The Protein Tyrosine Phosphatase 4A (PTP4A) family of three proteins, also known as Phosphatases of Regenerating Liver (PRLs), are dual specificity phosphatases that act as oncogenes in multiple cancer types. Specifically, PRL-3 has been identified as a potential cancer biomarker¹. PRL-3 expression was found to be upregulated in metastatic colorectal cancer in 2001². Since then, PRL-3 has been demonstrated to be implicated in progression and metastasis in gastric³, ovarian⁴, breast⁵, brain⁶, and prostate⁷ cancers, melanoma^{8,9}, and leukemias^{10,11}. Experimental evidence indicates that PRL-3 expression increases proliferation, migration, and invasion of cancer cells in vitro^{12,13,14} and enhances tumor growth and metastasis in mouse models^{2,15,} while PRL-3 knockdown significantly suppresses tumor formation and spread in vivo¹⁶. While overexpression of PRL-3 in tumors plays roles in inhibiting apoptosis, promoting epithelial to mesenchymal transition (EMT), and inducing migration, the mechanisms by which PRL-3 drives these processes and its physiological substrates remain unclear. The function of PRL-3 must be better defined if drugs targeting this protein are to be brought to the clinic.

The open questions regarding PRL-3, including its normal and cancerous function, localization, and substrate(s) are largely the result of insufficient tools to study this protein. The development of specific small molecule PRL-3 inhibitors has been difficult, as PRL proteins are highly homologous, and the PRL catalytic binding pocket is both shallow and hydrophobic″. Currently, the most frequently used PRL inhibitors are the PRL-3 Inhibitor I (Sigma P0108), Analog 3¹⁸, thienopyridone¹⁹, and JMS-05334²⁰ all of which inactivate PRLs via a redox reaction instead of directly binding with the protein's active site. Antibodies specific for PRL-3 have also proven difficult with most antibodies lacking specificity towards PRL-3 over other PRL proteins. An antibody that has been validated for specificity for PRL-3 over PRL-1 and PRL-2²″⁹ was raised against the linear form of PRL-3 and cannot be used for studies assessing the native protein. A humanized monoclonal antibody, PRL-3-zumab, was recently developed and is shown to specifically bind to PRL-3 and have anti-cancer effects in vivo²². The authors predict that PRL-3 is capable of being presented on the cell surface through exosomal secretion, allowing for the binding of PRL-3-zumab. This event stimulates Fc-receptor dependent interactions between PRL-3 positive cells and host immune effectors, activating classical antibody-mediated tumor clearance pathways leading to tumor cell death²². However, while PRL-3-zumab is currently in phase 2 clinical trial (NCT04118114) being tested against gastric and hepatocellular carcinomas, this antibody is not currently commercially available. Overall, research tools to study PRL-3 are lacking.

Nanobodies have recently emerged as immensely useful research tools and are likely to become useful therapeutics in a variety of diseases, including cancer^{23,24}. Nanobodies were discovered in dromedaries, such as camels, llamas, and alpacas. These animals produce both antibodies with typical structure and those with an atypical structure that lacks light chains but has a similar variable region (VHH region) to conventional antibodies²⁵. The lack of light chains causes formation of a longer complementary determining region-(CDR)3 with a secondary disulfide bond²⁶ to stabilize nanobody structure. This shape permits formation of convex shapes, allowing nanobodies to reach narrow, concave binding and activation sites on proteins that normal antibodies cannot²⁷. Other advantages of nanobodies include their small size at ˜15 kDa, suggesting they can penetrate cellular membranes, as well as their stability under stringent conditions, lack of immunogenicity, and a high specificity and affinity for their antigens²⁵.

There are many inherent properties of nanobodies that make them advantageous as therapeutics and specifically for cancer applications²⁸. The small size of nanobodies enables deep penetration in tumors where some can even cross the blood brain barrier²⁹, while maintaining low off-target effects³⁰. These properties, as well as the fact that nanobodies can withstand high temperatures, elevated pressure, non-physiological pHs and denaturants make them ideal candidates for studying proteins in multiple aspects and as therapeutic molecules²⁸. Nanobodies are being widely applied in diagnostics and therapies for many diseases. In 2019, the first nanobody therapeutic was approved by the FDA, Caplacizumab or Cablivi, which aids in accelerating platelet aggregation in acquired thrombotic thrombocytopenic purpura (aTTP), a disease that causes small blood clots throughout the body. In terms of cancer, there are over 18 on-going clinical trials currently studying the efficacy of nanobodies in a plethora of cancers²⁸.

Despite the research noted above, there remains a need for small molecule inhibitors for PRL-3.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

In some embodiments, the presently-disclosed subject matter includes an amino acid molecule comprising an amino acid sequence according to one or more of NB91 (SEQ ID NO: 1), NB 13 (SEQ ID NO: 2), NB90 (SEQ ID NO: 3), NB4 (SEQ ID NO: 4), NB7 (SEQ ID NO: 5), NB10 (SEQ ID NO: 6), NB16 (SEQ ID NO: 7), NB18 (SEQ ID NO: 8), NB29 (SEQ ID NO: 9), NB19 (SEQ ID NO: 10), NB84 (SEQ ID NO: 11), NB92 (SEQ ID NO: 12), NB23 (SEQ ID NO: 13), NB26 (SEQ ID NO: 14), NB28 (SEQ ID NO: 15), NB68 (SEQ ID NO: 16), or a variant thereof. In some embodiments, the molecule specifically binds to a protein tyrosine phosphatase 4A (PTP4A or PRL) protein. In some embodiments, the PRL is PRL-3.

Also provided herein, in some embodiments, is a composition comprising the amino acid molecule tagged with a compound for degrading a targeted PRL protein. In some embodiments, the compound for degrading the targeted PRL protein is an E3 ubiquitin ligase. In some embodiments, the amino acid molecule is further tagged with an immunotoxin. In some embodiments, the targeted PRL protein is PRL-3.

Further provided herein, in some embodiments, is a method of detecting a PRL protein, the method comprising contacting a sample with the amino acid molecule and detecting binding between the amino acid molecule and the PRL protein. In some embodiments, the PRL protein is PRL-3. In some embodiments, the method further includes quantifying the PRL protein using ELISA. In some embodiments, the method further includes identifying substrates of the PRL protein using immunoprecipitation. In some embodiments, the method further includes defining PRL protein localization using a cell-based assay. In some embodiments, the cell-based assay is immunofluorescence.

Still further provided herein, in some embodiments, is a method of targeting a cancer cell, the method comprising contacting the cell with the amino acid molecule.

Also provided herein, in some embodiments, is a method of treating cancer, the method comprising administering the composition to a subject in need thereof, where the cancer includes a cancer that expresses PRL-3. In some embodiments, the compound for degrading the targeted PRL protein is an E3 ubiquitin ligase. In some embodiments, the amino acid molecule is further tagged with an immunotoxin. In some embodiments, the cancer comprises a metastatic cancer. In some embodiments, the metastatic cancer is selected from the group comprising breast, prostate, colon, melanoma, leukemia, and combinations thereof.

Further provided herein, in some embodiments, is a method of making the amino acid molecule, the method comprising cloning a cDNA sequence of the amino acid molecule into a bacterial expression vector.

Further features and advantages of the presently-disclosed subject matter will become evident to those of ordinary skill in the art after a study of the description, figures, and non-limiting examples in this document.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIGS. 1A-B show a schematic and image illustrating isolation of PRL-3 specific nanobodies. (A) Schematic demonstrating the process completed from initial recombinant PRL-3 injection in alpacas to colony PCR to reach sequence identification of potential anti-PRL-3 nanobodies. (B) Amino acid sequence for 16 nanobodies that showed positive result in colony PCR and in sequencing with Eurofins primer pexR. NB91 was the most common sequence in our pool of clones, demonstrating necessary nanobody components with PelB (directs protein to the bacterial periplasm during expression), and expected complimentary determining regions. Each group of nanobodies is either the same sequence as NB91, differs by 1-2, 10-20, or 25+ amino acids.

FIGS. 2A-D show graphs illustrating that nanobodies are specific for PRL-3 over the other PRL family members. (A) ELISA utilizing 96-well plates coated with 100 ng of the PRLs, probed with 100 ng of each nanobody followed by 1:1000 TheTM His Tag Antibody (Genscript). ELISA of 100 ng of each (B) PRL-1, (C) PRL-2, (D) PRL-3 saturated with up to 200 ng of 16 nanobodies. ELISAs were developed for 1.5 minutes with 100 μl TMB 2-Component Microwell Peroxidase Substrate Kit (Seracare) and stopped with 100 μl 0.1N HCl, and read at 450 nm. All assays were completed with two technical replicates and repeated in two biological replicates.

FIGS. 3A-B show images illustrating that nanobodies can be used to immunoprecipitate overexpressed PRL-3. PRL-3 specific nanobodies coupled to superparamagnetic Dynabeads® M-270 Epoxy beads were used in immunoprecipitation assays with lysates from HEK293T cells transduced with 3×FLAG-PRL-1, -2 or -3. (A) All nanobodies pull down 3×FLAG-PRL-3 with little to no pull down of 3×FLAG-PRL-1 or 3×FLAG-PRL-2. (B) Successful nanobody coupling to Dynabeads in all groups was verified using an antibody against the C-terminal 6×His-tag present on each nanobody.

FIG. 4 shows an image illustrating that nanobodies are specific to PRL-3 in immunofluorescence assays. HCT116 colorectal cancer cells were transfected with A—CMV:GFP, CMV:GFP-PRL-1, CMV:GFP-PRL-2, or CMV:GFP-PRL-3 for 24 hours prior to cell fixation and permeabilization. Immunofluorescence assays were completed with 1:1000 1 mg/mL NB91 followed by 1:400 Alexa Fluor® 594-AffiniPure Goat Anti-Alpaca IgG, VHH domain and show nanobodies detect PRL-3 but not PRL-1 or PRL-2.

FIGS. 5A-D show images illustrating representative recombinant PRL protein purification. (A) PRL-1 was expressed and purified from BL21 DE3 Star bacterial cells using Ni-Column Chromatography, including cleavage of 6×His-tag. (B) PRL-2 was expressed and purified from BL21 DE3 Star bacterial cells using Ni-Column Chromatography, including cleavage of 6×His-tag. (C) PRL-3 was expressed and purified from BL21 DE3 Star bacterial cells using Ni-Column Chromatography, including cleavage of 6×His-tag. (D) PRL-1, -2, and -3 following Size Exclusion Chromatography on an AKTA Chromatography System and Superdex 200 Increase 10/300 GL Column and concentration of samples. L—ladder, IS—insoluble proteins, S—soluble proteins, FT—flowthrough, W—wash, E—elution, CP—column pass.

FIGS. 6A-B show images illustrating representative recombinant nanobody protein purification. (A) All nanobodies were purified using the same bacterial cell line, using Ni-Column Chromatography with two elution steps (30 mM and 250 mM Imidazole). L—ladder, FT —flowthrough, W—wash, E—elution. (B) Following Ni-Column, nanobodies underwent Size Exclusion Chromatography on an AKTA Chromatography System and Superdex 200 Increase 10/300 GL Column. Nanobody 13 is representative for all nanobodies in this study. C12-D04 denotes fraction number when eluted from the size exclusion column.

FIGS. 7A-G show graphs illustrating that nanobodies do not specifically inhibit the phosphatase activity of PRL-3. (A-G) Phosphatase activity of 2.5 μM each of (A) NB4, (B) NB10, (C) NB16, (D) NB19, (E) NB26, (F) NB84, and (G) NB91 incubated with recombinant PRL-1, -2, and -3 (2.5 μM) was measured using the EnzChek™ Phosphatase Assay Kit (ThermoFisher). The graphs demonstrate that phosphatase activity is not affected by the presence of any of the seven nanobodies. All assays were completed with six technical replicates and repeated in two biological replicates.

FIGS. 8A-B show images illustrating 3×FLAG-PRL immunoprecipitation controls. Controls demonstrating that the Dynabeads® M-270 Epoxy beads do not alter inherent (A) 3×FLAG-PRL-1, -2; or (B) 3×FLAG-PRL-3 binding compared to NB91. S—Supernatant, B—Beads, W—Wash.

FIGS. 9A-I show images illustrating total protein for HEK293T 3×FLAG-PRL IPs. (A-G) Total protein gels for all nanobody IPs with HEK293T 3×FLAG-PRL lysate, corresponding to IPs in FIGS. 3A-B. (H-I) Total protein gels for nanobody 91 control IPs with HEK293T 3×FLAG-PRL lysate, corresponding to IPs in FIGS. 7A-G.

FIGS. 10A-F show images illustrating six other nanobodies can detect overexpressed PRL-3 in fixed cells. HCT116 colorectal cancer cells were transfected with either CMV:GFP-PRL-1, -2, or -3 for 24 hours prior to fixation and permeabilization. (A-F) IFs were completed with 1:1000 1 mg/mL (A) nanobody 4, (B) nanobody 10, (C) nanobody 16, (D) nanobody 19, (E) nanobody 26, (F) nanobody 84, followed by 1:400 Alexa Fluor® 594-AffiniPure Goat Anti-Alpaca IgG, VHH domain (Jackson ImmunoResearch) and visualized using a Nikon MR confocal microscope.

FIGS. 11A-B show images and graphs illustrating that nanobody 19 stabilizes PRL-3 structure at two sites and destabilizes PRL-3 at one interaction point. (A) Nanobody 19 shows regions of both increased and decreased deuterium uptake with approximately 70% sequence coverage, with gray areas representing portions of PRL-3 where deuterium exchange was not detected. (B) Peptide 13-19 showed PRL-3 being deprotected following nanobody binding while peptides 56-64 and 132-146 showed decreases in deuterium uptake reflecting more protection by nanobody 19 on PRL-3 in these regions.

FIGS. 12A-B show images and graphs illustrating that nanobody 26 stabilizes PRL-3 structure at two sites and destabilizes PRL-3 at one interaction point. (A) Nanobody 26 shows regions of both increased and decreased deuterium uptake with approximately 70% sequence coverage, with gray areas representing portions of PRL-3 where deuterium exchange was not detected. (B) Peptide 13-19 showed PRL-3 being deprotected following nanobody binding while peptides 63-79 and 132-146 showed decreases in deuterium uptake reflecting more protection by nanobody 91 on PRL-3 in these regions.

FIGS. 13A-B show images and graphs illustrating that nanobody 91 stabilizes PRL-3 structure at two sites and destabilizes PRL-3 at one interaction point. (A) Nanobody 91 shows regions of both increased and decreased deuterium uptake with approximately 70% sequence coverage, with gray areas representing portions of PRL-3 where deuterium exchange was not detected. (B) Peptide 13-19 showed PRL-3 being deprotected following nanobody binding while peptides 56-79 and 132-146 showed decreases in deuterium uptake reflecting more protection by nanobody 91 on PRL-3 in these regions.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes an amino acid molecule or nanobody that is directed against and/or that can specifically bind to a protein tyrosine phosphatase 4A (PTP4A or PRL) protein. In some embodiments, the amino acid molecule is directed against and/or can specifically bind to PRL-1, PRL-2, or PRL-3. In some embodiments, the amino acid molecule is directed against and/or binds to the oncogenic phosphatase Protein Tyrosine Phosphatase 4A3 (PTP4A3 or PRL-3) with high specificity over other members of the same phosphatase family (e.g., PRL-1 and PRL-2). In some embodiments, the amino acid molecule includes one or more of NB91 (SEQ ID NO: 1), NB 13 (SEQ ID NO: 2), NB90 (SEQ ID NO: 3), NB4 (SEQ ID NO: 4), NB7 (SEQ ID NO: 5), NB10 (SEQ ID NO: 6), NB16 (SEQ ID NO: 7), NB18 (SEQ ID NO: 8), NB29 (SEQ ID NO: 9), NB19 (SEQ ID NO: 10), NB84 (SEQ ID NO: 11), NB92 (SEQ ID NO: 12), NB23 (SEQ ID NO: 13), NB26 (SEQ ID NO: 14), NB28 (SEQ ID NO: 15), NB68 (SEQ ID NO: 16), and/or variants thereof.

Also provided herein are methods of detecting a PRL protein. In some embodiments, the method includes contacting a sample with the amino acid molecule or nanobody as disclosed herein, and detecting binding between the amino acid molecule or nanobody and PRL protein. In some embodiments, the amino acid molecule or nanobody as disclosed herein is specific to PRL-3 over other over other members of the same phosphatase family. Accordingly, in some embodiments, the method includes specifically detecting PRL-3 protein with the amino acid molecule or nanobody disclosed herein. For example, in one embodiment, the method includes quantifying PRL-3 through ELISA. In another embodiment, the method includes identifying substrates of PRL-3 through immunoprecipitation. In a further embodiment, the method includes defining PRL-3 localization through a cell-based assay, such as, but not limited to, immunofluorescence. Without wishing to be bound by theory, it is believed that the amino acid molecules or nanobodys disclosed herein are the first anti-PRL-3 nanobodies in the PRL field capable of specifically detecting PRL-3 protein without overlapping with other family members (e.g., PRL-1 and PRL-2).

Also provided herein, in some embodiments, is a composition including one or more of the amino acid molecules or nanobodys as disclosed herein, and a compound for degrading a targeted PRL protein attached and/or tagged to the amino acid molecule. The composition may include any suitable compound for degrading the targeted PRL protein, such as, but not limited to, any suitable compound for degrading PRL-3 protein. For example, in one embodiment, the compound includes an E3 ubiquitin ligase. In another embodiment, the amino acid molecules or nanobodies tagged with an E3 ubiquitin ligase degrade PRL-3 in cells. Additionally or alternatively, in some embodiments, the composition includes an immunotoxin tagged to the amino acid molecules or nanobodies. In such embodiments, administration of the composition kills cells expressing high levels of PRL-3.

PRL-3 is not expressed by normal tissue, but is expressed very highly by aggressive and metastatic cancers, including, but not limited to, breast, prostate, colon, melanoma, and some leukemias. Moreover, PRL-3 have known roles in cancer progression (e.g., promoting metastasis) and are associated with poor prognosis. Accordingly, further provided herein are methods of targeting a cancer cell that expresses PRL-3 and/or expresses high levels of PRL-3. For example, in some embodiments, the method of targeting a cancer cell includes contacting the cell with the amino acid molecules or nanobodys as disclosed herein. In some embodiments, the method includes treating a cancer that expresses and/or expresses high levels of PRL-3, the method comprising administering the composition according to one or more of the embodiments disclosed herein to a subject in need thereof.

As compared to antibodies, the presently-disclosed subject matter makes use of amino acid molecules or nanobodies, which are more stable and smaller than antibodies, and can therefore enter into cells and will stay in the system longer. Additionally, nanobodies do not generate a host immune response, unlike antibodies, so would cause less side effects in treatment. They are also significantly less expensive to produce since they can be made in bacteria.

Still further provided herein are methods of making an amino acid molecule or composition as disclosed herein. In some embodiments, the method includes cloning the cDNA sequences into a bacterial expression vector (e.g., pMES4). Additionally or alternatively, in some embodiments, prior to cloning the cDNA sequences into a bacterial expression vector, the method includes inoculating alpacas with PRL proteins, collecting blood from the inoculated alpacas over a set period (e.g., several months), and isolating cDNA sequences from B-cells of the inoculated animals from the collected blood. In some embodiments, the cDNA sequences are fused with a C-terminal 6×His-tag before expression in the bacterial expression vector. For example, in one embodiment, the cDNA sequences are fused with a C-terminal 6×His-tag and expressed in BL21 Star (DE3) Chemically Competent Bacteria Cells by induction with 0.5 mM IPTG for 16 hours at 4 degrees Celsius.

In some embodiments, the method includes harvesting recombinant amino acid molecules or nanobody proteins by resuspending and lysing the bacterial cells. For example, in one embodiment, the method includes harvesting recombinant amino acid molecules or nanobody proteins by resuspending in 10 mL of lysis buffer [300 mM NaCl, 20 mM Tris pH 7.5, 10 mM Imidazole pH 8.0, 1:1000 protease inhibitor cocktail] per gram of cell pellet and lysing using a microfluidizer (Avestin, EmulsiFlex-05). In some embodiments, the method includes isolating the recombinant amino acid molecules or nanobody protein. For example, in one embodiment, the method includes isolating the recombinant amino acid molecules or nanobody protein using Ni-NTA Resin and eluting with increasing concentrations of elution buffer [300 mM NaCl, 20 mM Tris pH 7.5, and 250 mM Imidazole pH 8.0]. In some embodiments, the method includes further purifying the recombinant amino acid molecules or nanobodies. For example, in one embodiment, the method includes purifying the recombinant amino acid molecules or were further purified using a Superdex 10/300 on a GE AKTA in buffer containing 100 mM NaCl and 200 mM HEPES pH 7.5. In some embodiments, the purified fractions are then run on 4-20% Mini-PROTEAN TGX Stain-Free Gels, the purest fractions are pooled, concentrated together, and then flash frozen and stored at −80° C.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases, such as GENBANK® and SWISSPROT. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter is further illustrated by the following specific but non-limiting examples. The following examples may include compilations of data that are representative of data gathered at various times during the course of development and experimentation related to the present invention.

### EXAMPLES

The Phosphatase of Regenerating Liver 3 (PRL-3), is a dual specificity phosphatase that acts as an oncogene in an array of solid and liquid tumors. The development of specific small molecule PRL-3 inhibitors has proven difficult, as the three members of the PRL family are highly homologous (˜80%). This Example discusses the development of specific molecules that can differentiate PRL-3 from other family members in order to study protein functions and interactions. Alpaca-derived PRL-3 nanobodies were designed, purified, and tested for their specificity for PRL-3 over other PRL family members. Seven unique nanobodies were identified that specifically bind to PRL-3 over PRL-1 and PRL-2 in ELISA, immunoprecipitation, and immunofluorescence experiments.

Methods and Materials

Plasmids and Other Reagents

To generate protein for alpaca immunization, human PRL-3 cDNA was amplified with gene specific primers and cloned into the bacterial expression vector pET28b at NheI and XhoI restriction sites using T4 ligase. Recombinant PRL-1, -2, and -3 were made in the same fashion for all subsequent assays.

The 3×FLAG-tagged PRL mammalian expression plasmids were made by cloning PCR products of full length PRL-1, -2, or -3 human cDNA into p3×FLAG-CMV-14 expression vector (Sigma, E7908). Then 3×-FLAG-PRLs were cloned into plenti-CMV-puro (Addgene 17452) to make plenti-CMV-3×FLAG-PRL-puro constructs.

The GFP-tagged PRL overexpressing plasmids were made by cloning full length PRL-1, -2, or -3 gBlocks™ Gene Fragments (IDT) into the pcDNATM3.1 (−) (Invitrogen V79520) at BamHI and HindIII restriction sites. A GFP gBlock was subsequently cloned into each of the pCDNA3.1-PRL plasmids to generate CMV:GFP-PRL fusion constructs at NotI and BamHI restriction sites.

Production, Panning, and Sequencing of Nanobodies

Nanobodies were produced by the University of Kentucky Protein Core, as previously described³¹. Briefly, 100 μg of recombinant PRL-3 antigen was subcutaneously injected into alpacas once per week for six weeks to boost nanobody presence in the immune system. 3-5 days following the final injection 50 mL of alpaca blood was harvested to isolate peripheral blood lymphocytes by density gradient centrifugation. RNA was isolated and cDNA was synthesized using reverse transcriptase, to generate bacteriophage display cDNA library by cloning with restriction enzymes into the phage display vector pMES4 followed by the expression of the insert fused to gene III of the filamentous phage for the production of the phage solution. Two rounds of phage display utilizing this cDNA library yielded 32 potentially VHH positive clones that were analyzed for sequencing using the primer pEX-Rev (CAGGCTTTACACTTTATGCTTCCGGC). DNA sequences were translated using the ExPASy Bioinformatics Resource Portal Translate Tool (https://web.expasy.org/translate/) where they were analyzed for nanobody components including pelB sequence and 6×-His-tag followed by a stop codon. 16 of 32 clones embodied all of these components and were carried through to following experiments.

Cell Lines and Cell Culture

All human cell lines used in this study (HEK293T, HCT116) were authenticated by short tandem repeat (STR) profiling and tested for mycoplasma contamination prior to experiments. HEK293T (ATCC CRL-3216) and HCT116 (ATCC CCL-247) cells were grown in 1×DMEM (Thermofisher, 11965092). For all, media were supplemented with 10% heat-inactivated fetal bovine serum (R&D Systems, 511150H, Lot. H19109). Cells were cultured at 37° C. with 5% CO2. To overexpress the CMV:GFP-PRL and CMV:3×FLAG-PRL plasmids, cells were transfected using Lipofectamine 3000 (Thermofisher, L3000-015) following the manufacturer's protocol.

Protein Purification

pET28b-PRL and pMES4-Nanobody expression plasmids described above were transformed into and expressed using the One Shot BL21 Star DE3 bacterial cell line (Invitrogen, C601003) by stimulating induction with 0.5 mM IPTG (Fisher Scientific, BP175510) for 16 hours at 16° C. following a culture O.D.600 of 0.6. Cells were pelleted at 5,000 rpm for 15 minutes at 4° C. and resuspended in 10 mL of lysis buffer [300 mM NaCl (VWR BDH9286), 20 mM Tris pH 7.5, 10 mM Imidazole pH 8.0 (Sigma-Aldrich 12399), 1:1000 protease inhibitor cocktail (Sigma-Aldrich P8465)] per gram of cell pellet and lysed using a microfluidizer (Avestin, EmulsiFlex-05). Debris was pelleted at 18,000 rpm for 50 minutes at 4° C. and lysate was run over 1 mL columns (Biorad, 7321010) packed with Ni-NTA Resin (VWR, 786-940). PRLs were eluted with 2 mL of elution buffer (300 mM NaCl, 20 mM Tris pH 7.5, and 250 mM Imidazole pH 8.0). Nanobodies underwent two elution steps, the first with 30 mM Imidazole elution buffer, and the second with 250 mM Imidazole elution buffer. The N-terminal 6×-His tag on recombinant PRLs was cleaved using TEV protease (gift from Konstantin Korotkov), and samples were reapplied to Ni-NTA column to remove uncleaved protein as well as TEV. Recombinant nanobodies remained with their C-terminal 6×-His-tag intact. All samples underwent buffer exchange to remove imidazole (300 mM NaCl, 20 mM Tris pH 7.5) and were further purified using a Superdex 200 Increase 10/300 GL column (GE, 28990944) on an AKTA purification system in buffer containing 100 mM NaCl and 20 mM HEPES (Fisher Scientific, BP310-100) pH 7.5. Purification was verified by running samples on 4-20% Mini-PROTEAN TGX Stain-Free Gels (Biorad 4568094). The purest fractions were pooled, concentrated together, flash frozen on dry ice, and stored at −80° C.

ELISA for Nanobody/PRL Binding Specificity

Recombinant, purified, PRL-1, -2, and -3 were plated at 1 μg/mL (100μ1) in Sodium Bicarbonate Buffer [0.42 g Sodium Bicarbonate (Fisher Scientific, BP328-500) in 50 mL diH20] in Corning® 96 Well EIA/RIA Assay Microplates (Sigma, CLS3590) and incubated for 16-20 hours at 4° C. Plates were washed three times with 0.05% PBST and loaded with a blocking solution of 0.5% BSA (Fisher Scientific, BP9706100) in 0.1% PBST for 1 hour at room temperature. Blocking buffer was removed and nanobodies were diluted to 1 μg/mL, or designated concentration for dosing experiments, and incubated in wells for 1 hour at room temperature. Wells were washed 3 times in PBS and incubated with 1:1000 anti-His HRP antibody (GenScript, A00612, Lot. 19K001984), for 1 hour at room temperature. Plates were washed 3 times with PBS and developed with TMB 2-Component Microwell Peroxidase Substrate Kit (Seracare, 5120-0053). Reactions were stopped after 90 seconds with 0.1 N HCl (Fisher Scientific, A144500) and read on a Biotek Synergy Multi-mode Plate Reader at 450 nm. Controls included PRL only wells, to specify lack of a 6×-His-tag, nanobody and secondary only wells to specify the necessity of PRL presence for binding, and buffer only to provide evidence that sodium bicarbonate and BSA could not elucidate a colorimetric change. Raw data from all control wells was pooled for each plate, and experimental wells were normalized to controls by dividing individual wells by average control wells. Individual well readouts were then placed in Prism 7 in Grouped format Table, where values for two replicate experiments were graphed for Relative Absorbance at 450 nm compared to average of control wells.

Nanobody Coupling to Dynabeads

PRL-3 nanobodies were coupled to Dynabeads (Life Technologies, 14311D) for downstream 3×FLAG-PRL-3 immunoprecipitation following manufacturer's instructions. Briefly, 150 μg of nanobody protein supplemented with C1 buffer to 250 μL was added to 5 mg of Dynabeads® M-270 Epoxy beads after the beads were washed with 1 mL of C1 buffer. Then, 250 μL of C2 buffer was added to the beads and nanobody mixture to incubate on a rotator at room temperature overnight (16-24 hours). After removing the supernatant, the nanobody-coupled beads were washed subsequently with HB (0.05% Tween 20), and LB (0.05% Tween 20) buffer once, SB buffer shortly twice and SB buffer for 15 minutes once. Finally, the nanobody-coupled beads were resuspended in 500 μL SB buffer and stored at 4° C. prior to experimentation.

Immunoprecipitation of PRL-3 with Nanobody Coupled to Dynabeads

HEK293T cells (˜20 million) were lysed for 30 minutes with intermittent vortexing in Pierce IP lysis buffer (Thermo 87788) supplemented with 1% protease inhibitor cocktail (IP buffer) at 500 μl per 10 million cells and spun at 12,000 rpm for 10 minutes at 4° C. to pellet cell debris. Protein concentration was quantified using the Quick Start Bradford 1× Dye Reagent (Biorad, 5000205). 150 μL of nanobody-coupled beads were washed in 1 mL of PBS for 5 minutes, precipitated, then equilibrated in 500 μL of IP buffer for 5 minutes. 2.5 mg of total extracted protein was added to the balanced nanobody-beads complex for incubation with rocking at 4° C. overnight. After washing the beads-protein complex in cold PBS four times, 50 μL 2× Laemmli Sample Buffer (Biorad, 161-0737) with 2-Mercaptoethanol (Fisher Scientific, 034461-100) was added to the beads, the mixture was boiled at 95° C. for 10 minutes, and the supernatant was collected for western blot analysis.

Western Blot

30 μg of total protein for input or the pulldown supernatant was loaded into a 4-20% Mini-PROTEAN® TGX Stain-Free™ Protein Gels. Total protein was assessed through stain free imaging on Biorad ChemiTouch Imaging System, which allows use of total protein as the loading control. Protein was transferred onto PVDF membrane (Biorad, 162-0255) using the Trans-Blot Turbo Transfer System (Biorad 1704150). Membranes were blocked with 5% milk in 0.1% TBST for 1 hour and probed with one of the following antibodies at the designated dilution overnight at 4° C. 1:3000 Monoclonal ANTI-FLAG® M2 antibody (Sigma, F1804, Lot. SLBK1346V) or 1:1000 His Tag Antibody. Following three washes with 0.1% TBST, secondary HRP-conjugated anti-mouse IgG antibody (Cell Signaling, 7076S, Lot. 33) was added at 1:2500 for 1 hour and membranes were imaged using Clarity Western ECL Substrate (Biorad, 1705061).

Immunofluorescence in Fixed GFP-PRL Cells with Nanobodies

Transfected cells were plated at 5,000 cells per well in 96-well black glass bottomed plates (Cellvis, P96-1.5H-N). All solution exchanges and imaging occurred in the 96-well plate. 24 hours post-transfection cells were fixed in 4% paraformaldehyde (VWR, AAJ61899-AK) for 15 minutes, rinsed in PBS, permeabilized for 10 minutes in 1% 100× Triton (Sigma, X100-100), and rinsed in PBS. Blocking solution of 2% BSA in PBS was applied for 1 hour to all wells. All nanobodies were diluted to 1 mg/ml in blocking solution, and further diluted 1:1000 and incubated with the cells for 1 hour at room temperature followed by five PBS washes. Detection was carried out using an anti-alpaca IgG VHH conjugated to Alexa Fluor-594 (Jackson ImmunoResearch, 128-585-232) diluted 1:400 in blocking solution, and counterstained with Hoechst, 1:1000 dilution (ThermoFisher, H3570). All wells were washed in PBS five times prior to imaging. Images were acquired with a Nikon MR confocal using the 40× water objective. Images were processed in Adobe Photoshop 2020 to both increase image brightness, overlay the 405 (Hoescht), 488 (GFP-PRL-3), and 561 (Nanobodies) channels. Channels were pseudocolored by RGB channels.

Phosphatase Assay

2.5 μM of recombinant PRL-1, -2, or -3 was mixed with 2.5 μM of each nanobody in black 384-well plates (Thermo Scientific, 164564), and incubated at room temperature for 1 hour in Reaction Buffer (20 mM Tris, 150 mM NaCl). Following incubation, the recombinant protein mixtures were combined with 12.5 μM diFMUP (Life Technologies, E12020), added to 384-well plates, and incubated for 20 minutes in the dark at room temperature. Fluorescence intensities were measured on a Biotek Synergy Multi-mode Plate Reader at 360 nm/460 nm excitation and emission receptively. Raw values for non-substrate containing controls were averaged and subtracted from values of wells incubated with substrate to remove background fluorescence. Raw values were transferred to Prism 7 in Grouped format where two replicate experiments were combined for final data processing.

Results

Alpaca Derived Anti-PRL-3 Nanobodies Exhibit Varying Amino Acid Sequences

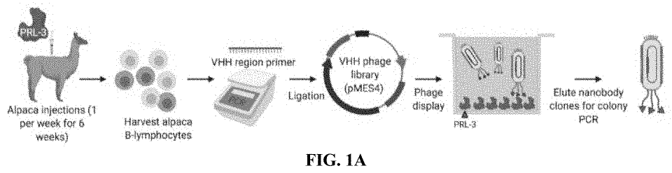

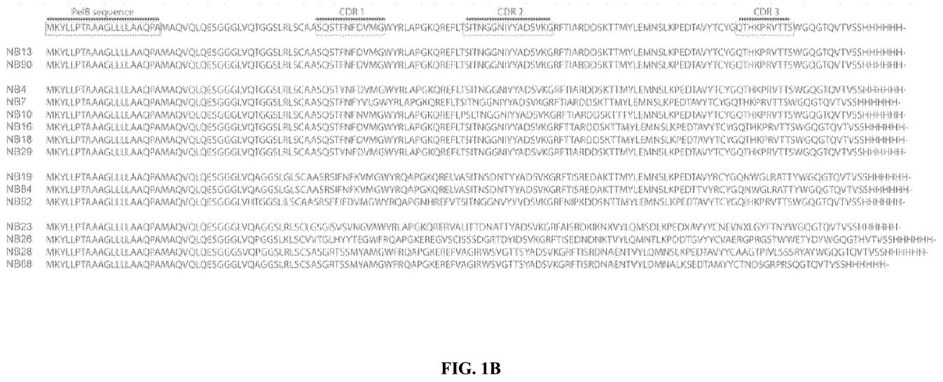

Human recombinant PRL-3 protein was injected into alpaca and single-domain antibodies, hereafter referred to as nanobodies, were harvested six weeks later and developed into a cDNA library, as diagramed in FIG. 1A. Bacteriophage display panning of the library against recombinant PRL-3 and subsequent sequencing of the enriched clones identified 32 potential nanobodies, of which only 16 nanobodies contained a complete N-terminal PelB sequence, C-terminal 6×-His tag, and stop codon, and were without any undetermined amino acids (FIG. 1B).

The anti-PRL-3 nanobody sequences were aligned to one another, and the putative high affinity binding regions were identified. Sequences of nanobodies 91, 90, and 13 were identical throughout and were the most frequently recurrent; therefore, nanobody 91 was utilized as the standard anti-PRL-3 nanobody throughout this Example. Nanobodies have been clustered based on their similarity to one another and the number of amino acid alterations or insertions based on nanobody 91. These include four groups; 0, 1-2, 10-20, and 25+ amino acid changes when compared to nanobody 91. Potential complimentary determining regions for these anti-PRL-3 nanobodies are proposed (FIG. 1B) based on known structures of other nanobodies interacting with antigen.

Anti-PRL-3 Nanobodies are Specific for PRL-3 Over Other PRL Family Members in Protein Assays

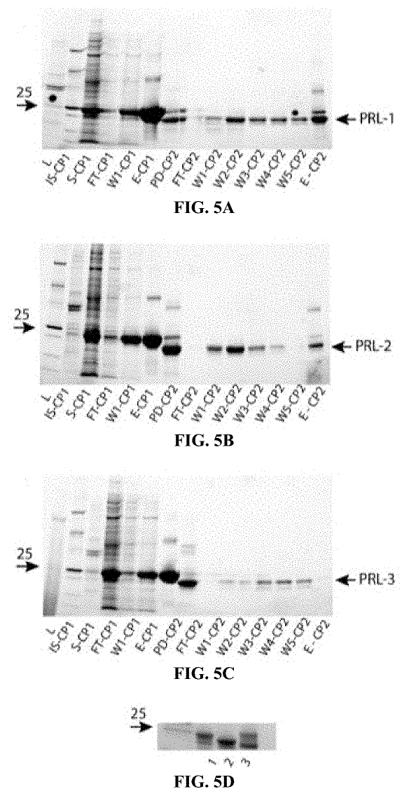

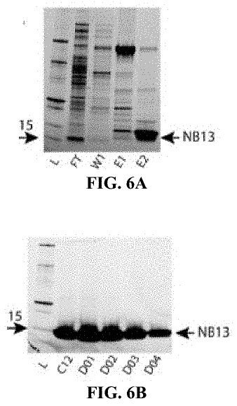

PRL-1 and PRL-2 have 79% and 76% amino acid sequence homology to PRL-3¹⁷, respectively, which has made identification of specific small molecules and antibodies difficult. An indirect ELISA method was used to test the specificity of the anti-PRL-3 nanobodies towards PRL-3 over other PRL family members. Nanobodies and PRLs were purified from BL21 DE3 Star E. coli (FIGS. 5A-6B), using nickel and size exclusion chromatography. The N-terminal 6×-His tag was cleaved from purified PRL proteins, while the C-terminal 6×-His-tag was left intact on nanobodies. PRL proteins were plated, and nanobody binding was detected through the addition of a secondary anti-His-HRP conjugated antibody.

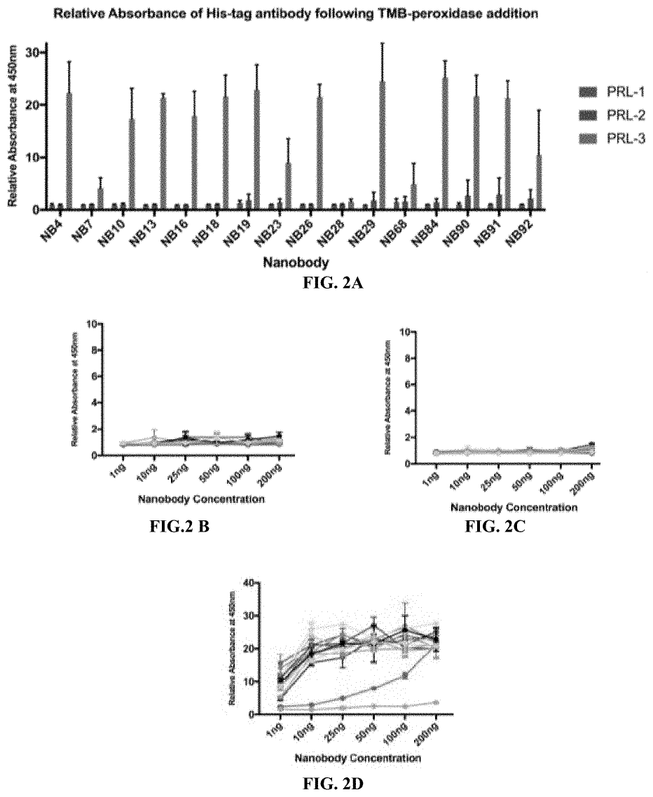

All 16 nanobodies had a greater affinity for PRL-3 over PRL-1 and PRL-2 (FIG. 2A), with most anti-PRL-3 nanobodies lacking any binding with PRL-1 or PRL-2 protein even in saturating conditions (FIGS. 2B-C). Nanobodies with the same amino acid sequences had comparable binding to PRL-3. Five nanobodies produced less of a colorimetric shift following binding to PRL-3 either due to poor expression in E. coli or low affinity for PRL-3 (nanobodies 23 and 28). Further studies focused on seven nanobodies (4, 10, 16, 19, 26, 84, and 91) with strong PRL-3 affinity and unique sequence both in the complimentary determining and framework regions.

Anti-PRL-3 Nanobodies do not Inhibit the Phosphatase Activity of PRL-3

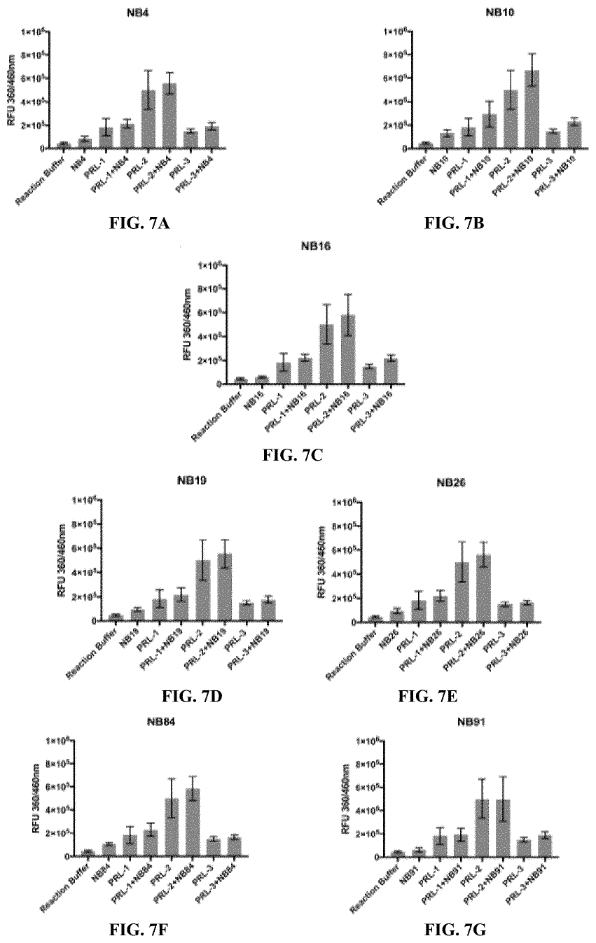

PRL-3 is a known oncogene, so specific targeting of PRL-3 to prevent function is very desirable. To determine if the nanobodies were capable of blocking PRL-3 phosphatase activity in in vitro assays, the ability of PRL-3/nanobody complexes to dephosphorylate a generic phosphorylated substrate, 6,8-Difluoro-4-Methylumbelliferyl Phosphate (diFMUP), was examined. Cleavage of a phosphate from diFMUP can induce a fluorescent signal at an excitation of 360 nm and emission at 460 mn, which can be quantified. However, there was no significant difference in fluorescence between the PRL-3 in complex with nanobody compared to PRL-3 alone (FIGS. 7A-G), suggesting that the nanobody does not occlude the PRL-3 active site. However, with a molecular weight of 292, diFMUP is very small in comparison to potential PRL-3 protein substrates—whether the nanobodies might block PRL-3/substrate interaction at the active site to prevent PRL-3 mediated dephosphorylation events remains to be determined.

Anti-PRL-3 Nanobodies Immunoprecipitated PRL-3 but not PRL-1 or PRL-2

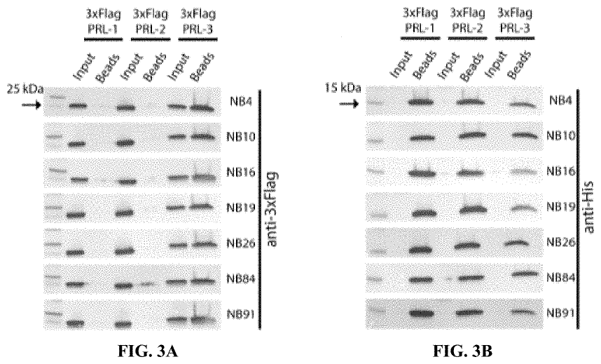

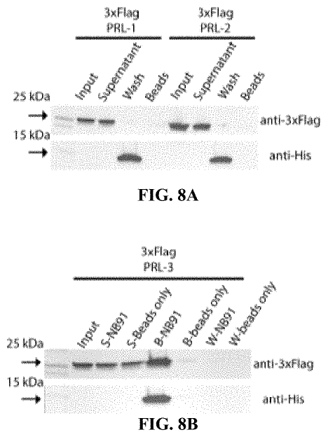

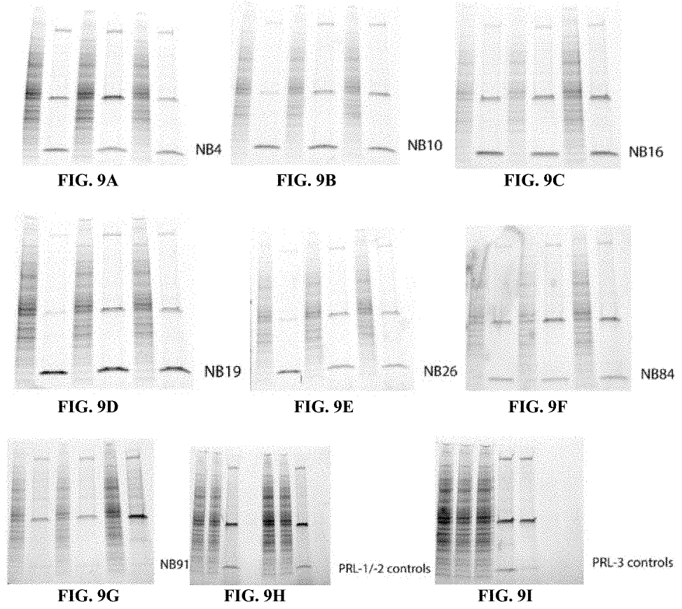

PRL-3 substrates remain largely undefined, in part due to insufficient tools for cell-based studies. The current commercially available PRL-3 antibodies have not been extensively validated for specificity towards PRL-3 over other PRLs. To address this, the ability of the anti-PRL-3 nanobodies to identify PRL-3 protein in cell lysate was tested. Anti-PRL-3 nanobodies were coupled to superparamagnetic Dynabeads® M-270 Epoxy beads and used in immunoprecipitation of HEK293T cells expressing either 3×FLAG-PRL-1, -2, or -3. All nanobodies selectively pulled-down 3×FLAG-PRL-3 over PRL-1 and PRL-2, as assessed by anti-FLAG immunoblot (FIGS. 7A and 9A-I). Some nanobodies were more specific to PRL-3 than others; nanobodies 4, 16, 19, and 84 pulled down small amounts of 3×FLAG-PRL-1 and/or PRL-2 (FIG. 3A). Successful nanobody coupling to Dynabeads was confirmed by the presence of 6×-His-tag in all samples (FIG. 3B). Beads alone do not immunoprecipitate 3×FLAG-PRL-3 (FIGS. 8A-B), indicating that the beads do not play a role in terms of immunoprecipitation. In total, anti-PRL-3 nanobodies 10, 26, and 91 can be used to specifically immunoprecipitate PRL-3, with no binding to PRL-1 or PRL-2.

Anti-PRL-3 Nanobodies Specifically Detect PRL-3 in Fixed Cells in Immunofluorescence Assays

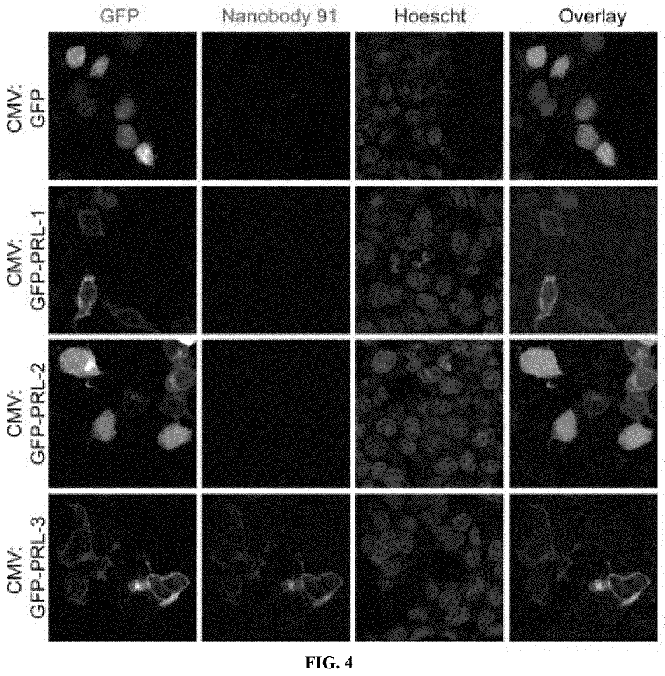

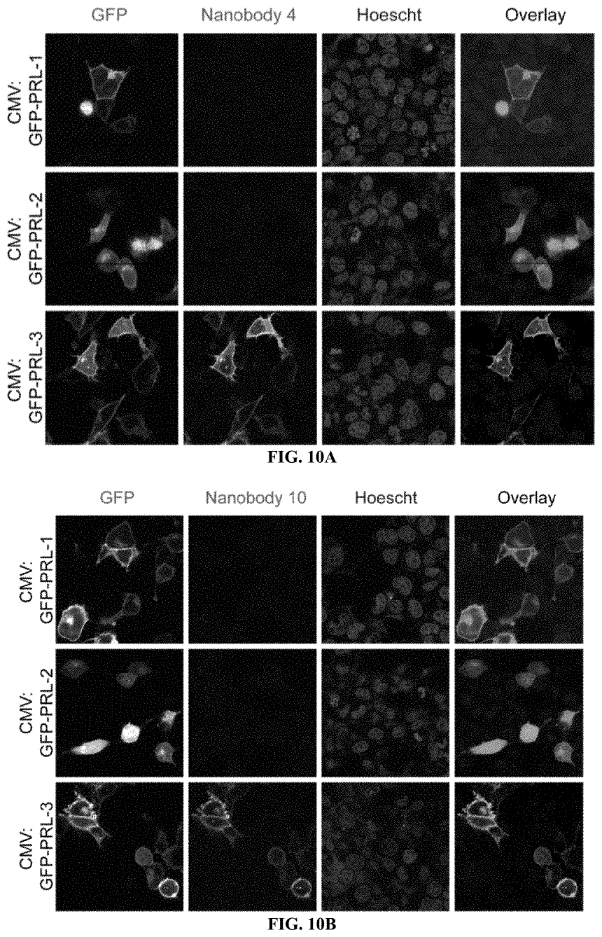

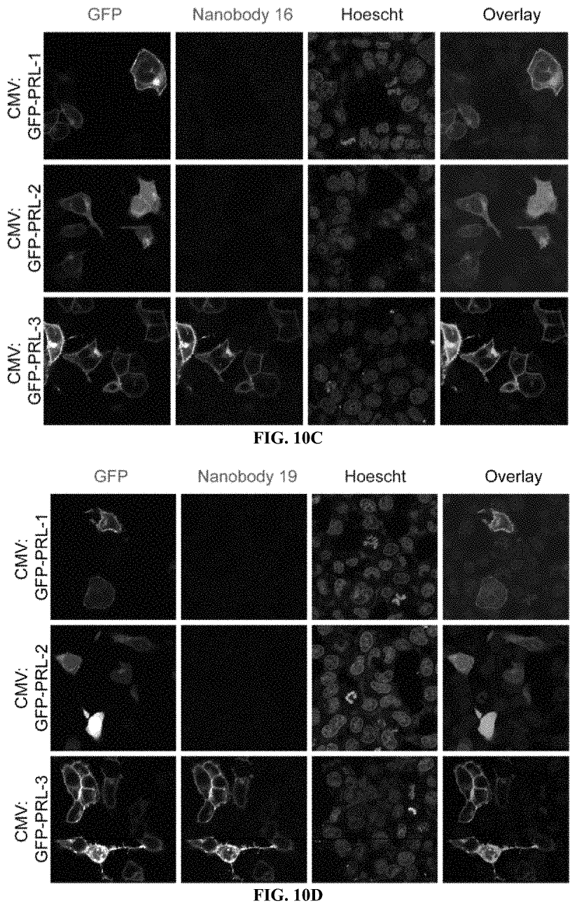

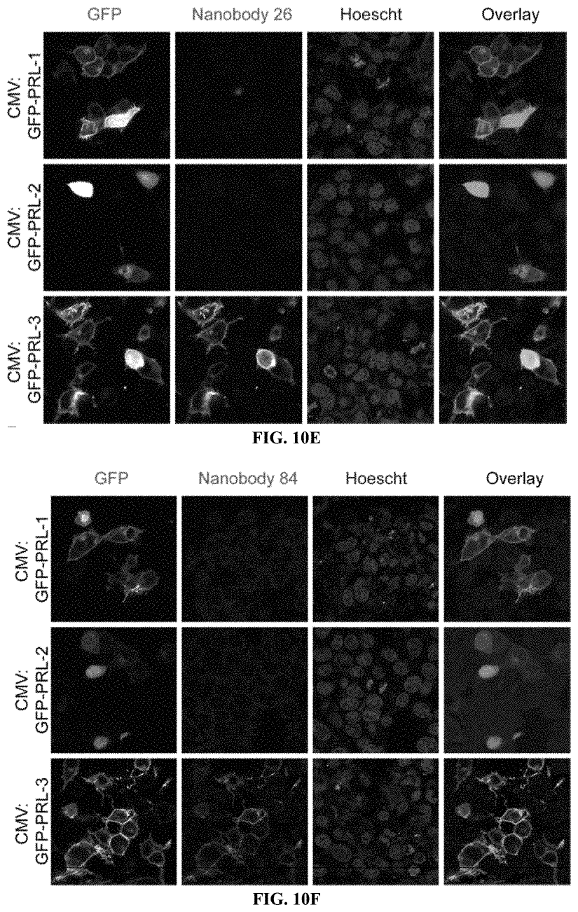

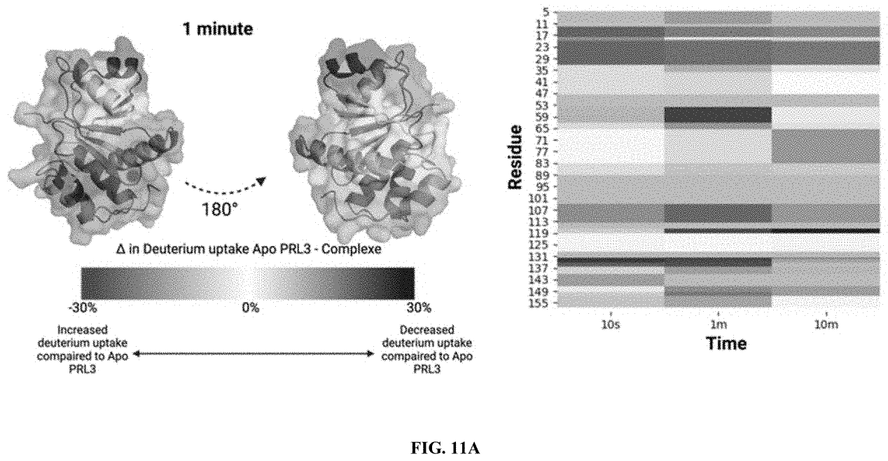

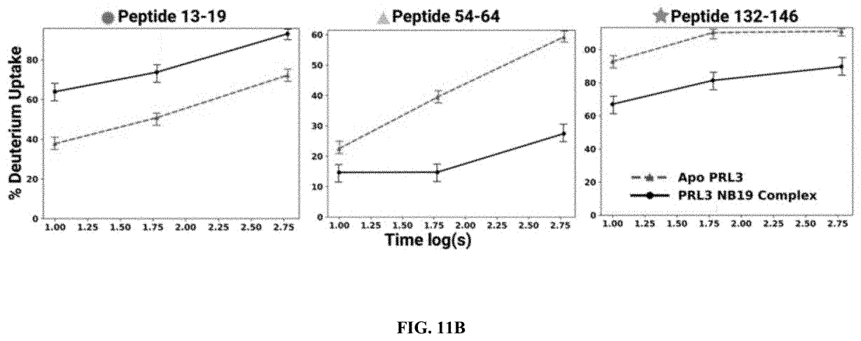

The next goal was to determine if nanobodies could specifically identify PRL-3 in fixed cells, which can be useful for studies involving PRL-3 localization and trafficking. The colon cancer cell line HCT116 was transfected with CMV:GFP-PRL constructs in order to visualize the PRLs and determine the extent to which the anti-PRL-3 nanobodies co-localize with each of them. Nanobody 91 completely co-localized with GFP-PRL-3, which was seen at both at the plasma membrane and in the nucleus (FIG. 4), as previously described^{32,33}. This nanobody did not bind with GFP-PRL-1, GFP-PRL-2, or the GFP control. All anti-PRL-3 nanobodies tested were similarly specific for PRL-3 over PRL-1 and PRL-2, although some nanobodies exhibited a higher non-specific background signal (FIGS. 10A-F).

### DISCUSSION

Tools to specifically study the members of the PRL family have continually lacked since their initial discovery approximately 20 years ago. In cancers that overexpress PRL-3, understanding how to specifically target this phosphatase has proved difficult. While PRL antibodies and inhibitors exist, the identification and characterization of the important roles that PRL-3 plays in tumor progression are not well understood. JMS-053 is termed a PRL-3 allosteric inhibitor that is equipotent for other PRL family members including PRL-1 and PRL-2²⁰. Therefore, in cellular and in vivo models, all three PRLs would be inhibited by MS-053. PRL-3 specific nanobodies can bind to PRL-3 without interacting with PRL-1 or PRL-2, making this tool much more specific.

PRL-3-zumab and PRL-3 nanobodies are similar in that they both have CDR regions and heavy chains, and have been shown to be specific to PRL-3 over PRL-1 and PRL-2 in vitro^{22,34}. However, nanobodies carry advantages over their conventional antibody counterparts in general and in terms of PRL-3. So far, PRL-3-zumab has been applied as an extracellular reagent, as it is hypothesized to bind PRL-3 on the cell surface to induce an immune response to kill cancer cells²². In contrast, the nanobodies discussed herein show promise to act as an intracellular reagent by recognizing PRL-3 in immunofluorescence experiments. Nanobodies have been used to study functional aspects of proteins in similar fashions such as sub-cellular localization and trafficking³⁵. The 10-fold decrease in size compared to conventional antibodies gives nanobodies an advantage when penetrating the cell membrane, a process that has been engineered by multiple groups to deliver nanobodies to their antigen^{36,37}.

While PRL-3-zumab is specific to PRL-3, the PRL-3 nanobodies are the first reagents to be used in immunoprecipitation experiments to identify PRL-3 binding partners. Nanobodies have been greatly demonstrated to work as chaperones in structural studies³¹, due to their decreased size and increased stability. Conventional antibodies are large, glycosylated, and multi-domain proteins, making them not nearly as suitable for applications such as X-ray crystallography. Nanobodies have been useful in both the crystallization and neutralization of proteins involved in disease such as the SARS-CoV-2 Spike protein³⁸ and are widely being used as a potential therapeutic in cancer39-41

In summary, this Example has finally begun to answer the question as to how researchers can specifically study members of the PRL phosphatase family in vitro. These are the first nanobodies that have been designed against PRL-3 and they can detect PRL-3 in both purified protein assays and in human cells. Importantly, it was also found that as they stand on their own, these nanobodies do not inhibit PRL-3 phosphatase activity. Revealing this new tool, and the capabilities it has in studying the mechanisms PRL-3 acts through functionally trafficking show great promise for future studies and developments. We have begun the fill the niche and need in developing PRL-3 inhibitors and tools for use in research and in clinic.

### Example 2

PRL-3 is an oncogenic phosphatase across multiple cancer types, including colon, ovarian, melanoma, breast, and leukemia. While there is increasing interest in developing PRL-3 inhibitors for use in cancer research and treatment, there have been several issues in designing small molecule inhibitors for PRL-3, such as a shallow, negative charged active site, homology between the PRL-3 active site and other protein tyrosine phosphatases, and a high degree of overall homology between PRL-3 and family members PRL-1 and PRL-2.

Nanobodies have recently emerged as an immensely useful research tool and show promise as a cancer therapeutic. Nanobodies are small, at ˜15 kD and lack light chains, allowing them to fit into spaces on target proteins that conventional antibodies cannot normally reach. Other advantages of nanobodies include their stability under stringent conditions, lack of immunogenicity, ability to permeate the cell, and a high specificity and affinity for their antigens.

Using full-length PRL-3 protein as an immunogen in alpaca, phage display technology was used to identify alpaca nanobodies that had high affinity for PRL-3 through subtractive panning. 18 unique nanobodies were identified through sequencing; 14 of these were able to be expressed in bacteria and purified. The binding specificity of the nanobodies to PRL-3 over PRL-1 and PRL-2 was determined through an indirect ELISA assay, which showed that that 12 out of 14 anti-PRL-3 nanobodies bound PRL-3 significantly better than PRL-1 or PRL-2 (˜25× times higher binding affinity to PRL-3, p<0.0001). Several nanobodies that stabilize PRL-3 structure were also identified using a Differential Scanning Fluorescence (DSF) assays to analyze shifts in the melting temperature of PRL-3 following binding, with the ultimate goal of developing PRL-3/nanobody crystal structures to identify nanobody binding sites and find PRL-3 active site binders. Additionally, the 6×-Histidine-tagged nanobodies were found to be useful for PRL-3 western blot, immunoprecipitation, and immunofluorescence, and mCherry-tagged nanobodies (chromobodies) allow for analysis of PRL-3 trafficking.

Nanobodies were also tested for their ability to inhibit the phosphatase activity of PRL-3, using both purified protein and functional in vitro assays. The results showed that several nanobodies decreased PRL-3 phosphatase activity. Overall, this Example describes both a novel research tool that can be used to gain insight into the structure and function of PRL-3 in normal and cancer cells, and a potentially new biologic inhibitor of PRL-3 that functions with high specificity and potency.

### Example 3

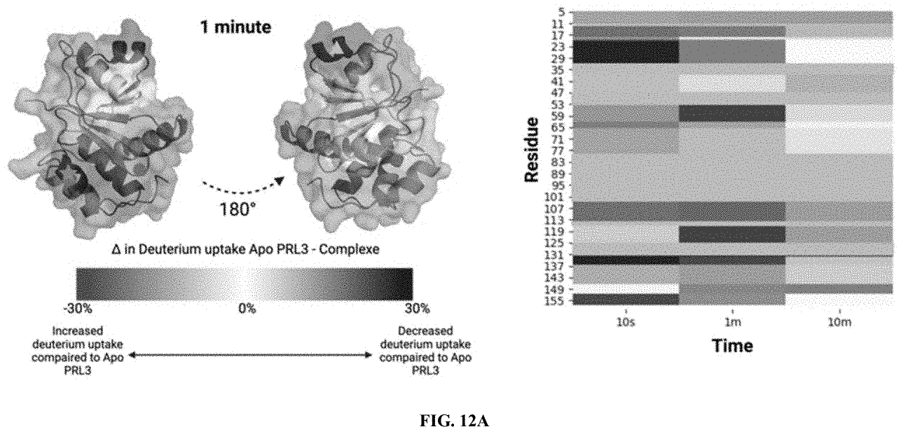

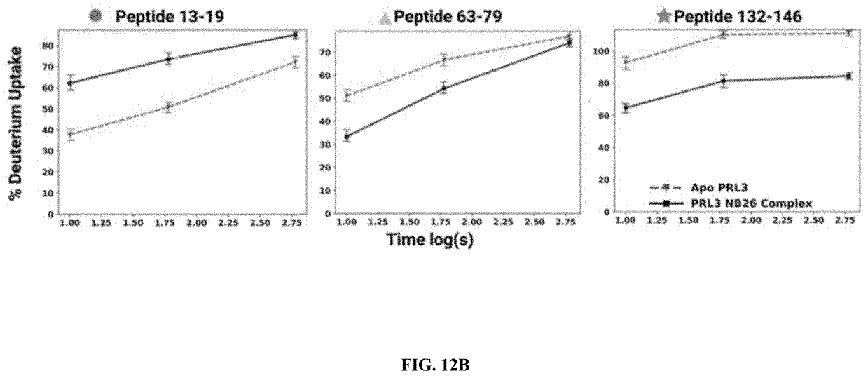

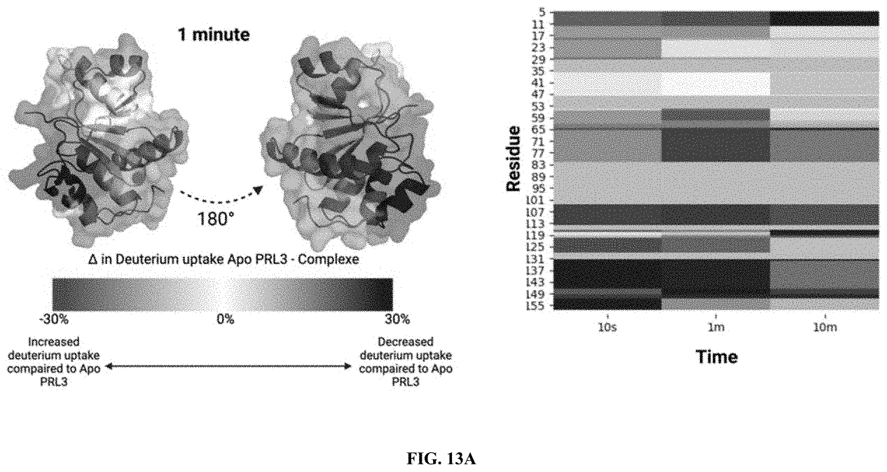

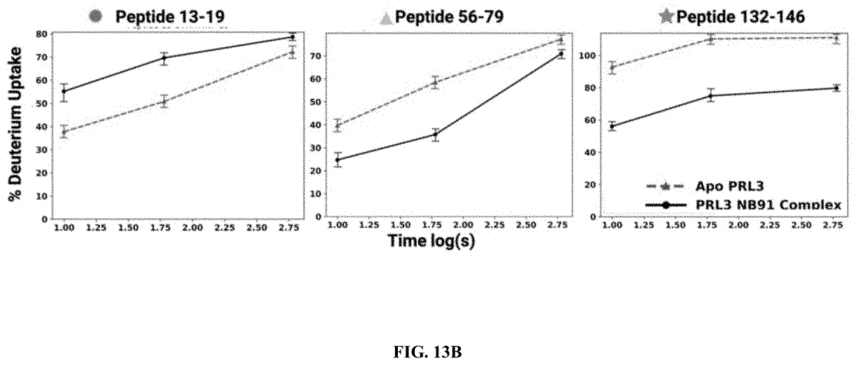

Referring to FIGS. 11A-13B, the data shown therein demonstrates which areas of PRL-3 are protected from heavy water (D₂0) when bound to each nanobody, giving insight as to where these nanobodies specifically bind on the PRL-3 protein. More specifically, FIGS. 11A-B are directed to nanobody 19, FIGS. 12A-B are directed to nanobody 26, and FIGS. 13A-B are directed to nanobody 91.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1 Tonks, N. K. Protein tyrosine phosphatases—from housekeeping enzymes to master regulators of signal transduction. FEBS J 280, 346-378, doi:10.1111/febs.12077 (2013).
2 Saha, S. et al. A phosphatase associated with metastasis of colorectal cancer. Science 294, 1343-1346, doi:10.1126/science.1065817 (2001).
3 Dai, N., Lu, A. P., Shou, C. C. & Li, J. Y. Expression of phosphatase regenerating liver 3 is an independent prognostic indicator for gastric cancer. World J Gastroenterol 15, 1499-1505, doi:10.3748/wjg.15.1499 (2009).
4 Ren, T. et al. Prognostic significance of phosphatase of regenerating liver-3 expression in ovarian cancer. Pathol Oncol Res 15, 555-560, doi:10.1007/s12253-009-9153-1 (2009).
5 den Hollander, P. et al. Phosphatase PTP4A3 Promotes Triple-Negative Breast Cancer Growth and Predicts Poor Patient Survival. Cancer Res 76, 1942-1953, doi:10.1158/0008-5472.CAN-14-0673 (2016).
6 Wang, L. et al. PTP4A3 is a target for inhibition of cell proliferatin, migration and invasion through Akt/mTOR signaling pathway in glioblastoma under the regulation of miR-137. Brain Res 1646, 441-450, doi:10.1016/j.brainres.2016.06.026 (2016).
7 Vandsemb, E. N. et al. Phosphatase of regenerating liver 3 (PRL-3) is overexpressed in human prostate cancer tissue and promotes growth and migration. J Transl Med 14, 71, doi:10.1186/s12967-016-0830-z (2016).
8 Fang, X. Y. et al. PRL-3 Promotes the Malignant Progression of Melanoma via Triggering Dephosphorylation and Cytoplasmic Localization of NHERF1. J Invest Dermatol 135, 2273-2282, doi:10.1038/jid.2015.154 (2015).
9 Johansson, J. A. et al. PRL3-DDX21 Transcriptional Control of Endolysosomal Genes Restricts Melanocyte Stem Cell Differentiation. Dev Cell 54, 317-332 e319, doi:10.1016/j.devcel.2020.06.013 (2020).
10 Wei, M., Haney, M. G., Rivas, D. R. & Blackburn, J. S. Protein tyrosine phosphatase 4A3 (PTP4A3/PRL-3) drives migration and progression of T-cell acute lymphoblastic leukemia in vitro and in vivo. Oncogenesis 9, 6, doi:10.1038/s41389-020-0192-5 (2020).
11 Zhou, J. et al. Phosphatase of regenerating liver-3 is regulated by signal transducer and activator of transcription 3 in acute myeloid leukemia. Exp Hematol 42, 1041-1052 e1041-1042, doi:10.1016/j.exphem.2014.08.001 (2014).
12 Al-Aidaroos, A. Q. et al. Metastasis-associated PRL-3 induces EGFR activation and addiction in cancer cells. J Clin Invest 123, 3459-3471, doi:10.1172/JCI66824 (2013).
13 Peng, L. et al. PRL-3 promotes the motility, invasion, and metastasis of LoVo colon cancer cells through PRL-3-integrin beta1-ERK1/2 and -MMP2 signaling. Mol Cancer 8, 110, doi:10.1186/1476-4598-8-110 (2009).
14 Radke, I. et al. Expression of PRL-3 regulates proliferation and invasion of breast cancer cells in vitro. Arch Gynecol Obstet 296, 1153-1160, doi:10.1007/s00404-017-4542-2 (2017).
15 Zeng, Q., Hong, W. & Tan, Y. H. Mouse PRL-2 and PRL-3, two potentially prenylated protein tyrosine phosphatases homologous to PRL-1. Biochem Biophys Res Commun 244, 421-427, doi:10.1006/bbrc.1998.8291 (1998).
Zimmerman, M. W., Homanics, G. E. & Lazo, J. S. Targeted deletion of the metastasis-associated phosphatase Ptp4a3 (PRL-3) suppresses murine colon cancer. PLoS One 8, e58300, doi:10.1371/journal.pone.0058300 (2013).
17 Wei, M., Korotkov, K. V. & Blackburn, J. S. Targeting phosphatases of regenerating liver (PRLs) in cancer. Pharmacol Ther 190, 128-138, doi:10.1016/j.pharmthera.2018.05.014 (2018).
18 Hoeger, B., Diether, M., Ballester, P. J. & Kohn, M. Biochemical evaluation of virtual screening methods reveals a cell-active inhibitor of the cancer-promoting phosphatases of regenerating liver. Eur J Med Chem 88, 89-100, doi:10.1016/j.ejmech.2014.08.060 (2014).
19 Daouti, S. et al. A selective phosphatase of regenerating liver phosphatase inhibitor suppresses tumor cell anchorage-independent growth by a novel mechanism involving p130Cas cleavage. Cancer Res 68, 1162-1169, doi:10.1158/0008-5472.CAN-07-2349 (2008).
20 McQueeney, K. E. et al. Targeting ovarian cancer and endothelium with an allosteric PTP4A3 phosphatase inhibitor. Oncotarget 9, 8223-8240, doi:10.18632/oncotarget.23787 (2018).
21 Ooki, A. et al. Phosphatase of regenerating liver-3 as a convergent therapeutic target for lymph node metastasis in esophageal squamous cell carcinoma. Int J Cancer 127, 543-554, doi:10.1002/ijc.25082 (2010).
22 Thura, M. et al. PRL3-zumab as an immunotherapy to inhibit tumors expressing PRL3 oncoprotein. Nat Commun 10, 2484, doi:10.1038/s41467-019-10127-x (2019).
23 Allegra, A. et al. Nanobodies and Cancer: Current Status and New Perspectives. Cancer Invest 36, 221-237, doi:10.1080/07357907.2018.1458858 (2018).
24 Bannas, P., Hambach, J. & Koch-Nolte, F. Nanobodies and Nanobody-Based Human Heavy Chain Antibodies As Antitumor Therapeutics. Front Immunol 8, 1603, doi:10.3389/fimmu.2017.01603 (2017).
25 Muyldermans, S. Single domain camel antibodies: current status. J Biotechnol 74, 277-302, doi:10.1016/s1389-0352(01)00021-6 (2001).
26 Krah, S. et al. Single-domain antibodies for biomedical applications. Immunopharmacol Immunotoxicol 38, 21-28, doi:10.3109/08923973.2015.1102934 (2016).
27 Schmitz, K. R., Bagchi, A., Roovers, R. C., van Bergen en Henegouwen, P. M. & Ferguson, K. M. Structural evaluation of EGFR inhibition mechanisms for nanobodies/VHH domains. Structure 21, 1214-1224, doi:10.1016/j.str.2013.05.008 (2013).
28 Yang, E. Y. & Shah, K. Nanobodies: Next Generation of Cancer Diagnostics and Therapeutics. Front Oncol 10, 1182, doi:10.3389/fonc.2020.01182 (2020).
29 Muruganandam, A., Tanha, J., Narang, S. & Stanimirovic, D. Selection of phage-displayed llama single-domain antibodies that transmigrate across human blood-brain barrier endothelium. FASEB J 16, 240-242, doi:10.1096/fj.01-0343fje (2002).
30 Muyldermans, S., Atarhouch, T., Saldanha, J., Barbosa, J. A. & Hamers, R. Sequence and structure of VH domain from naturally occurring camel heavy chain immunoglobulins lacking light chains. Protein Eng 7, 1129-1135, doi:10.1093/protein/7.9.1129 (1994).
31 Chow, K. M. et al. Immunization of Alpacas (Lama pacos) with Protein Antigens and Production of Antigen-specific Single Domain Antibodies. J Vis Exp, doi:10.3791/58471 (2019).
32 Lian, S. et al. PRL-3 promotes telomere deprotection and chromosomal instability. Nucleic Acids Res 45, 6546-6571, doi:10.1093/nar/gkx392 (2017).
33 Zeng, Q. et al. Prenylation-dependent association of protein-tyrosine phosphatases PRL-1, -2, and -3 with the plasma membrane and the early endosome. J Biol Chem 275, 21444-21452, doi:10.1074/jbc.M000453200 (2000).
34 Thura, M. et al. PRL3-zumab, a first-in-class humanized antibody for cancer therapy. JCI Insight 1, e87607, doi:10.1172/jci.insight.87607 (2016).
35 Schiavon, C. R. et al. Actin chromobody imaging reveals sub-organellar actin dynamics. Nat Methods 17, 917-921, doi:10.1038/s41592-020-0926-5 (2020).
36 Herce, H. D. et al. Cell-permeable nanobodies for targeted immunolabelling and antigen manipulation in living cells. Nat Chem 9, 762-771, doi:10.1038/nchem.2811 (2017).
37 Woods, J. Selection of Functional Intracellular Nanobodies. SLAS Discov 24, 703-713, doi:10.1177/2472555219853235 (2019).
38 Huo, J. et al. Neutralizing nanobodies bind SARS-CoV-2 spike RBD and block interaction with ACE2. Nat Struct Mol Biol 27, 846-854, doi:10.1038/s41594-020-0469-6 (2020).
39 Tabtimmai, L. et al. Cell-penetrable nanobodies (transbodies) that inhibit the tyrosine kinase activity of EGFR leading to the impediment of human lung adenocarcinoma cell motility and survival. J Cell Biochem 120, 18077-18087, doi:10.1002/jcb.29111 (2019).
40 Dekempeneer, Y. et al. Labeling of Anti-HER2 Nanobodies with Astatine-211: Optimization and the Effect of Different Coupling Reagents on Their in Vivo Behavior. Mol Pharm 16, 3524-3533, doi:10.1021/acs.molpharmaceut.9b00354 (2019).
41 Schriewer, L. et al. Nanobody-based CD38-specific heavy chain antibodies induce killing of multiple myeloma and other hematological malignancies. Theranostics 10, 2645-2658, doi:10.7150/thno.38533 (2020).

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. An amino acid molecule comprising an amino acid sequence according to one or more of NB91 (SEQ ID NO: 1), NB 13 (SEQ ID NO: 2), NB90 (SEQ ID NO: 3), NB4 (SEQ ID NO: 4), NB7 (SEQ ID NO: 5), NB10 (SEQ ID NO: 6), NB16 (SEQ ID NO: 7), NB18 (SEQ ID NO: 8), NB29 (SEQ ID NO: 9), NB19 (SEQ ID NO: 10), NB84 (SEQ ID NO: 11), NB92 (SEQ ID NO: 12), NB23 (SEQ ID NO: 13), NB26 (SEQ ID NO: 14), NB28 (SEQ ID NO: 15), NB68 (SEQ ID NO: 16), or a variant thereof.

2. The amino acid molecule of claim 1, wherein the molecule specifically binds to a protein tyrosine phosphatase 4A (PTP4A or PRL) protein.

3. The amino acid molecule of claim 2, wherein the PRL is PRL-3.

4. A composition comprising the amino acid molecule of claim 1 tagged with a compound for degrading a targeted PRL protein.

5. The composition of claim 4, wherein the compound for degrading the targeted PRL protein is an E3 ubiquitin ligase.

6. The composition of claim 4, wherein the amino acid molecule is further tagged with an immunotoxin.

7. The composition of claim 4, wherein the targeted PRL protein is PRL-3.

8. A method of detecting a PRL protein, the method comprising:
contacting a sample with the amino acid molecule of claim 1; and
detecting binding between the amino acid molecule and the PRL protein.

9. The method of claim 8, wherein the PRL protein is PRL-3.

10. The method of claim 8, further comprising quantifying the PRL protein using ELISA.

11. The method of claim 8, further comprising identifying substrates of the PRL protein using immunoprecipitation.

12. The method of claim 8, further comprising defining PRL protein localization using a cell-based assay.

13. The method of claim 12, wherein the cell-based assay is immunofluorescence.

14. A method of targeting a cancer cell, the method comprising contacting the cell with the amino acid molecule of claim 3.

15. A method of treating cancer, the method comprising:
administering the composition of claim 4 to a subject in need thereof;
wherein the cancer includes a cancer that expresses PRL-3.

16. The method of claim 15, wherein the compound for degrading the targeted PRL protein is an E3 ubiquitin ligase.

17. The method of claim 15, wherein the amino acid molecule is further tagged with an immunotoxin.

18. The method of claim 15, wherein the cancer comprises a metastatic cancer.

19. The method of claim 18, wherein the metastatic cancer is selected from the group consisting of breast, prostate, colon, melanoma, leukemia, and combinations thereof.

20. A method of making the amino acid molecule of claim 1, the method comprising cloning a cDNA sequence of the amino acid molecule into a bacterial expression vector.


SEQUENCE LISTING (16 sequences)

SEQ ID NO: 1 - protein, 146 aa, Artificial Sequence
  Nanobody 91
NKYLLPTAAAGLLLLAAQPAMAQVQLQESGGGLVQTGGSLRLSCAASQSTFNFDVMGWYRLAPGKQREFLTSITNGGNIYYADSVKGRFTIARDDSKTTMYLEMNSLKPEDTAVYTCYGQTHKPRVTTSWGQGTQVTVSSHHHHHH

SEQ ID NO: 2 - protein, 146 aa, Artificial Sequence
  Nanobody 13
MKYLLPTAAAGLLLLAAQPAMAQVQLQESGGGLVQTGGSLRLSCAASQSTFNFDVMGWYRLAPGKQREFLTSITNGGNIYYADSVKGRFTIARDDSKTTMYLEMNSLKPEDTAVYTCYGQTHKPRVTTSWGQGTQVTVSSHHHHHH

SEQ ID NO: 3 - protein, 146 aa, Artificial Sequence; identical to SEQ ID NO: 2
  Nanobody 90

SEQ ID NO: 4 - protein, 146 aa, Artificial Sequence
  Nanobody 4
MKYLLPTAAAGLLLLAAQPAMAQVQLQESGGGLVQTGGSLRLSCAASQSTYNFDVMGWYRLAPGKQREFLTSITNGGNIYYADSVKGRFTIARDDSKTTMYLEMNSLKPEDTAVYTCYGQTHKPRVTTSWGQGTQVTVSSHHHHHH

SEQ ID NO: 5 - protein, 146 aa, Artificial Sequence
  Nanobody 7
MKYLLPTAAAGLLLLAAQPAMAQVQLQESGGGLVQTGGSLRLSCAASQSTFNFYVLGWYRLAPGKQREFLTSITNGGNIYYADSVKGRFTIARDDSKTTMYLEMNSLKPEDTAVYTCYGQTHKPRVTTSWGQGTQVTVSSHHHHHH

SEQ ID NO: 6 - protein, 146 aa, Artificial Sequence
  Nanobody 10
MKYLLPTAAAGLLLLAAQPAMAQVQLQESGGGLVQTGGSLRLSCAASQSTFNFNVMGWYRLAPGKQREFLPSLTNGGNIYYADSVKGRFTIARDDSKTTTYLEMNSLKPEDTAVYTCYGQTHKPRVTTSWGQGTQVTVSSHHHHHH

SEQ ID NO: 7 - protein, 146 aa, Artificial Sequence
  Nanobody 16
MKYLLPTAAAGLLLLAAQPAMAQVQLQESGGGLVQTGGSLRLSCAASQSTFNFDVMGWYRLAPGKQREFLTSITNGGNIYYADSVKGRFTTARDDSKTTMYLEMNSLKPEDTAVYTCYGQTHKPRVTTSWGQGTQVTVSSHHHHHH

SEQ ID NO: 8 - protein, 145 aa, Artificial Sequence
  Nanobody 18
MKYLLPTAAAGLLLAAQPAMAQVQLQESGGGLVQTGGSLRLSCAASQSTFNFDVMGWYRLAPGKQREFLTSITNGGNIYYADSVKGRFTTARDDSKTTMYLEMNSLKPEDTAVYTCYGQTHKPRVTTSWGQGTQVTVSSHHHHHH

SEQ ID NO: 9 - protein, 146 aa, Artificial Sequence; identical to SEQ ID NO: 4
  Nanobody 29

SEQ ID NO: 10 - protein, 146 aa, Artificial Sequence
  Nanobody 19
MKYLLPTAAAGLLLLAAQPAMAQVQLQESGGGLVQAGGSLGLSCAASRSIFNFKVMGWYRQAPGKQRELVASITNSDNTYYADSVKGRFTISREDAKTTMYLEMNSLKPEDTAVYRCYGQNWGLRATTYWGQGTQVTVSSHHHHHH

SEQ ID NO: 11 - protein, 146 aa, Artificial Sequence
  Nanobody 84
MKYLLPTAAAGLLLLAAQPAMAQVQLQESGGGLVQAGGSLGLSCAASRSIFNFKVMGWYRQAPGKQRELVASITNSDNTYYADSVKGRFTISREDAKTTMYLEMNSLKPEDTTVYRCYGQNWGLRATTYWGQGTQVTVSSHHHHHH

SEQ ID NO: 12 - protein, 146 aa, Artificial Sequence
  Nanobody 92
MKYLLPTAAAGLLLLAAQPAMAQVQLQESGGGLVHTGGSLILSCAASRSFFIFDVMGWYRQAPGNHREFVTSITNGGNVYYVDSVKGRFNIPKDDSNTTMYLEMNSLKPEDTAVYTCYGQIHKPRVTTSWGQGTQVTVSSHHHHHH

SEQ ID NO: 13 - protein, 146 aa, Artificial Sequence
  Nanobody 23
  misc_feature (99)..(99): Xaa can be any naturally occurring amino acid
  misc_feature (112)..(112): Xaa can be any naturally occurring amino acid
  misc_feature (122)..(122): Xaa can be any naturally occurring amino acid
MKYLLPTAAAGLLLLAAQPAMAQVQLQESGGGLVQAGGSLRLSCLGSGISVSVNGVAWYRLAPGKQRERVALITTDNATTYADSVKGRFAISRDKIKNXVYLQMSDLKPEDXAVYYCNEVNXLGYFTNYWGQGTQVTVSSHHHHHH

SEQ ID NO: 14 - protein, 150 aa, Artificial Sequence
  Nanobody 26
MKYLLPTAAAGLLLLAAQPAMAQVQLQESGGGLVQPGGSLKLSCVVTGLHYYTEGWFRQAPGKEREGVSCISSSDGRTDYIDSVKGRFTISEDNDNKTVYLQMNTLKPDDTGVYYCVAERGPRGSTWWETYDYWGQGTHVTVSSHHHHHH

SEQ ID NO: 15 - protein, 150 aa, Artificial Sequence
  Nanobody 28
MKYLLPTAAAGLLLLAAQPAMAQVQLQESGGGSVQPGGSLRLSCSASGRTSSMYAMGWFRQAPGKEREFVAGIRWSVGTTSYADSVKGRFTISRDNAENTVYLQMNSLKPEDTAVYYVAAGTPIVLSSSRYAYWGQGTQVTVSSHHHHHH

SEQ ID NO: 16 - protein, 142 aa, Artificial Sequence
  Nanobody 68
MKYLLPTAAAGLLLLAAQPAMAQVQLQESGGGLVQAGGSLRLSCSASGRTSSMYAMGWFRQAPGKEREFVAGIRWSVGTTSYADSVKGRFTISRDNAENTVYLDMNALKSEDTAMYYCTNDSGRPRSQGTQVTVSSHHHHHH